(19) United States
(12) Patent Application Publication
(10) Pub. No.: US 2016/0263322 A1
(43) Pub. Date: Sep. 15, 2016

(54) Injection Devices

(71) Applicant: Owen Mumford Ltd., Oxford, Oxfordshire (GB)

(72) Inventor: Toby COWE, Oxford (GB)

(21) Appl. No.: 15/031,446

(22) PCT Filed: Oct. 21, 2014
(86) PCT No.: PCT/GB2014/053136

§ 371 Date: Apr. 22, 2016

(30) Foreign Application Priority Data
Oct. 24, 2013 (GB) 1318817.2

(51) Int. Cl. A61M 5/20; A61M 5/31; A61M 5/315; A61M 5/24

(52) U.S. Cl. CPC A61M 5/20; A61M 5/2429; A61M 5/3148; A61M 5/3155; A61M 5/3158; A61M 5/31593; A61M 2005/2073; A61M 2005/2403; A61M 2005/3128

(57) ABSTRACT

An injection device comprises a housing (5), an axially moveable dose delivery mechanism (10), an actuation button (20) for imparting movement to said delivery mechanism (10), and a dose setting arrangement (30) to select a dose volume. The dose setting arrangement (30) comprises a threaded member (31) arranged to engage a formation (9) on, or fixed relative to, the housing (5) such that axial movement relative to the housing (5) results in relative angular movement of the threaded member (31). The dose setting arrangement also includes a stop (9a); fixed relative to the housing, and arranged to cooperate with the threaded member (31) limiting axial movement thereof. Complementary engagement features (16, 36) are provided which are moveable between an engaged position in which relative axial movement of the delivery mechanism (10) and threaded member (31) is prevented; and a disengaged position in which the threaded member (31) may be moved relative to the delivery mechanism (10) to adjust the dose. In use, forward movement of the actuation button (20) moves the engagement features (16, 36) into the engaged position.

### FIELD OF INVENTION

The present invention relates to injection devices and, in particular but not exclusively, to injection devices for injecting medicament in multiple doses of an adjustable magnitude.

### BACKGROUND OF INVENTION

Injection devices provide a convenient way to administer medicament and are typically provided in the form of a pen-injector in which a cartridge or syringe containing therapeutic material, for example insulin, is received in (or formed by) a pen-type body.

Such injection devices are generally provided with a drug-delivery mechanism arranged to expel a dose from the cartridge or syringe in response to a user pressing an actuation button. In many such injection devices, there is also provided a dose-setting arrangement which enables the user to select a dose volume prior to actuation. Such dose mechanisms are generally arranged to administer a plurality of repeatable single metered doses with the volume of each single dose selectable by means of the dose-setting arrangement.

In most known injection devices the delivery mechanism includes a plunger which is progressively advanced during use in the forward axial direction. The plunger typically engages a bung within the cartridge or syringe which is moved forwarded to dispel a dose from the cartridge. An alternative dose delivery mechanism has been proposed in the applicant's earlier UK patent application GB1217765.5 which removes the need for a plunger (which is particularly advantageous in reducing the size of the injection device). Rather, the delivery mechanism is arranged to draw medicament from the cartridge into a conduit by negative pressure and subsequently discharge a metered dose from the conduit via the delivery needle. One convenient way of arranging a mechanism utilising this principle is to provide two relatively moveable conduit sections which are arranged to be telescoped together to change the volume of the combined conduit.

Embodiments of the invention seek to provide a dose-setting arrangement which may for example be conveniently used with a variety of drug delivery mechanisms and which may, for example, enable a user to easily and quickly adjust the required dose volume.

### STATEMENTS OF INVENTION

According to a first aspect of the invention, there is provided an injection apparatus for use with a cartridge or syringe to deliver a plurality of doses therefrom, the injection device comprising:
a housing;
an axially moveable dose delivery mechanism arranged to deliver a dose from said cartridge or syringe;
an actuation button for imparting movement, directly or indirectly, to said delivery mechanism; and
a dose setting arrangement to select a dose volume, wherein the dose setting arrangement comprises:
   a threaded member arranged to engage a formation on, or fixed relative to, the housing such that axial movement relative to the housing results in relative angular movement of the threaded member;
   a stop, fixed relative to the housing, and arranged to cooperate with the threaded member limiting axial movement thereof; and
   complementary engagement features moveable between an engaged position in which relative axial movement of the delivery mechanism and threaded member is prevented; and a disengaged position in which the threaded member may be moved relative to the delivery mechanism to adjust the dose; and wherein
   forward movement of the actuation button moves the engagement features into the engaged position.

According to a further embodiment of the invention there is provided an injection device apparatus for use with a cartridge or syringe to deliver a plurality of doses therefrom, the injection device comprising:
a housing;
a dose delivery mechanism arranged to deliver a dose from said cartridge or syringe;
an actuation button for imparting movement to said delivery mechanism; and
a dose setting arrangement to select a dose volume; wherein the actuation movement is transferred from the actuation button to the dose setting arrangement and resulting axial movement of the dose setting arrangement causes inter-engagement with the dose delivery mechanism to cause delivery movement thereof.

Thus, embodiments of the invention provide an arrangement in which the dose setting arrangement may be disengaged from the delivery mechanism to enable a does to be set and may automatically engage the delivery mechanism to control the dose delivered.

The threaded member may be annular. The threaded member may be positioned to radially surround the delivery mechanism. Advantageously, such an arrangement enables the dose setting arrangement to be provided with minimal impact upon the overall size (and particularly the axial length) of the injection device.

The stop may be any suitable form for limiting the rotational/axial movement of the threaded member. For example, the stop could simply be a blind end to the thread on the housing. The stop could alternatively or additionally include an abutment surface on, or associated with, the housing. A complementary abutment surface may be provided on the threaded member.

The complementary engagement features may include at least one radially moveable finger associated with one of the delivery mechanism or the dose setting arrangement. The complementary engagement features may comprise a plurality of axially spaced projections, for example annular ribs, associated with the other of the delivery mechanism or the dose-setting arrangement. The projections may be provided on a shaft of the delivery mechanism. The at least one moveable finger may be attached to the dose-setting mechanism. The moveable finger may be arranged to selectively engage the projections. For example, the finger may be radially deflected towards the projections so as to be moved into engagement.

Whilst the complementary engagement features axially couple the threaded member and delivery mechanism, the delivery mechanism and threaded member may be free to rotate angularly relative to one another. Accordingly, when the delivery mechanism moves axially relative to the housing and the engagement features are in engagement, the threaded member of the dose-setting arrangement will, as a result of the axial movement, be rotated by the formation on the housing.

The dose-setting mechanism may further comprise an intermediate member disposed between the threaded member and the delivery mechanism. The intermediate member may be a sleeve. The intermediate member may comprise the complementary engagement features for engaging the corresponding features on the delivery mechanism. For example, the intermediate member may be provided with the at least one radially moveable finger.

The intermediate member may be angularly fixed relative to the threaded member. The intermediate member may be axially moveable relative to the threaded member. For example, the intermediate member may be provided with a limited range of axial movement relative to the threaded member. The axial movement between the intermediate member and threaded member may be arranged to move the complementary engagement feature to the engaged position. For example, one of the complementary engagement features or threaded member may be provided with a cam surface. The cam surface may be arranged to be moved into contact with a surface the threaded member and to result in displacement of the engagement feature (for example, deflecting the engagement feature radially inward). For example, the engagement feature may be a finger which includes a cam surface on its radially outer surface and, wherein the cam surface is engaged by a surface of the threaded member so as to deflect the finger radially inwardly to engage the delivery mechanism.

In some embodiments, the delivery mechanism may comprise a plunger. Accordingly, the plunger may be provided with complementary engagement features (for example, axially spaced circumferential ribs) which are engaged by the complementary engagement features of the dose-setting mechanism.

In other embodiments the delivery mechanism may comprise a chamber or piston which is axially displaceable to alter the volume within the delivery mechanism. Accordingly, the complementary engagement features may be provided on the chamber or piston. For example, the complementary engagement features may comprise axially spaced circumferential ribs on the radially outer surface of either the chamber or piston member. Typically, one of the piston or chamber is axially fixed with respect to the housing and the complementary engagement may, therefore, be provided on the axially moveable one of the chamber or piston.

The delivery mechanism may be arranged to draw medicament from the cartridge or syringe by negative pressure. The delivery mechanism may be disposed forward of the cartridge or syringe. The injection device may further comprise a drive member. The drive member may extend axially between the actuation button and the delivery mechanism. The actuation button will typically be provided proximal to or at a rearward end of the injection device. The delivery mechanism may be provided at or proximal to the forward end of the injection device.

The actuating movement from the actuating button to the delivery mechanism may be transmitted via the dose selection arrangement. In particular, the actuating movement may be coupled via the complementary engagement features. The drive mechanism may transmit the actuating movement from the actuation button to the dose setting arrangement.

The drive member may be axially coupled to the dose-setting arrangement. The drive member may be angularly coupled to the dose-setting arrangement. For example, the drive member may be coupled to the intermediate member of the dose-setting arrangement. The drive member may be tubular and may be arranged to extend around the cartridge or syringe.

The actuation button may be connected to the drive member via a clutch. As such, the actuating button may be selectively angularly coupled to the drive member. Forward movement of the actuation button may be arranged to disengage the clutch. As such, the drive member may be able to freely rotate with the dose-setting arrangement when a user depresses the actuation button to deliver a dose. Additionally, or alternatively, the actuation button may be able to selectively positively engage the drive member such that a rotational force applied to the actuation button may be transmitted via the drive member to the dose-setting arrangement to enable a user to adjust the dose.

Whilst the invention has been described above, it extends to any inventive combination of feature set out above or in the following description and/or drawings.

### BRIEF DESCRIPTION OF DRAWINGS

A preferred embodiment of the invention will now be described in detail, by way of example only, and with reference to the accompanying drawings in which:

FIG. 1 is a schematic three-dimensional view of a pen injection device according to an embodiment of the invention;

FIG. 2 is a schematic cross-sectional view of the pen injection device according to an embodiment of the invention;

FIG. 3 is an exploded three-dimensional view of the pen injection device according to an embodiment of the invention;

FIG. 4 is a detailed view of the dose-setting arrangement of the pen injection device according to an embodiment of the invention; and

FIG. 5 is a partial cross-sectional view of the dose-setting arrangement according to an embodiment of the invention.

### DETAILED DESCRIPTION OF AN EMBODIMENT

Front as used herein will be understood to refer to the end of the injection device assembly (or components thereof) which, in use, are closest to the delivery needle end of the injection device (i.e. the end which is intended to be proximal to the skin). Rear as used herein will be understood to refer to the end of the injection device assembly (or components thereof) which, in use, are furthest from the delivery needle end of the pen injection device (i.e. the end which is intended to faces away from the skin). Likewise, forward and rearward will be understood to refer to the directions orientated towards the front and rear of the injection device assembly.

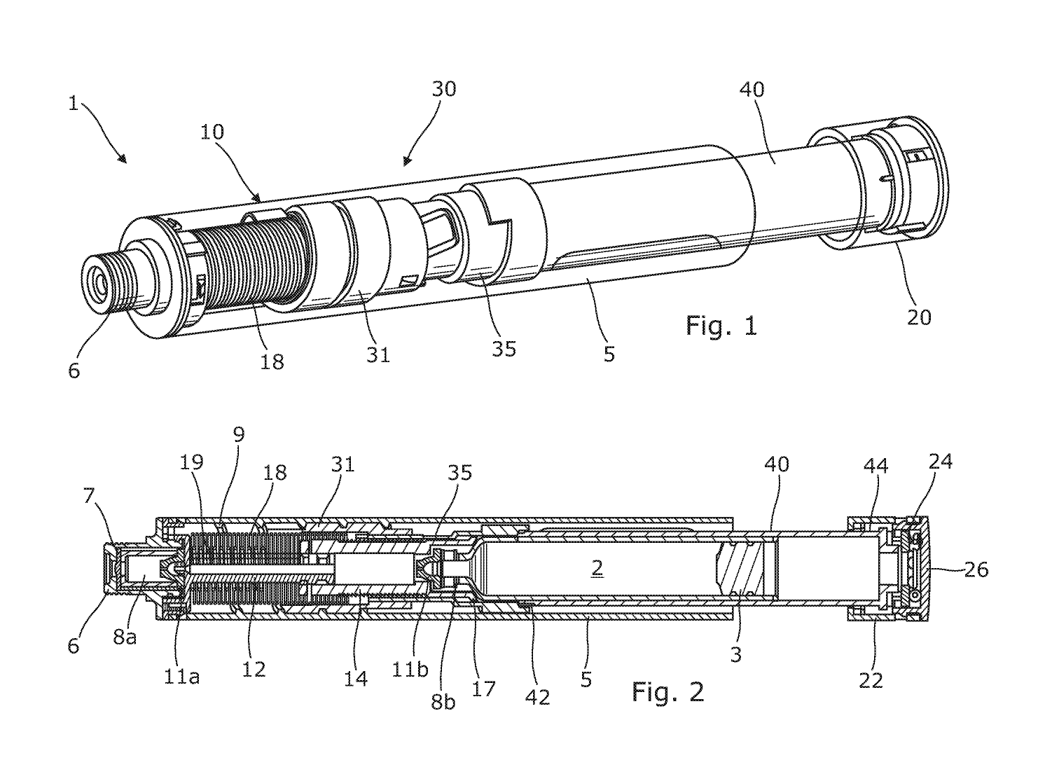

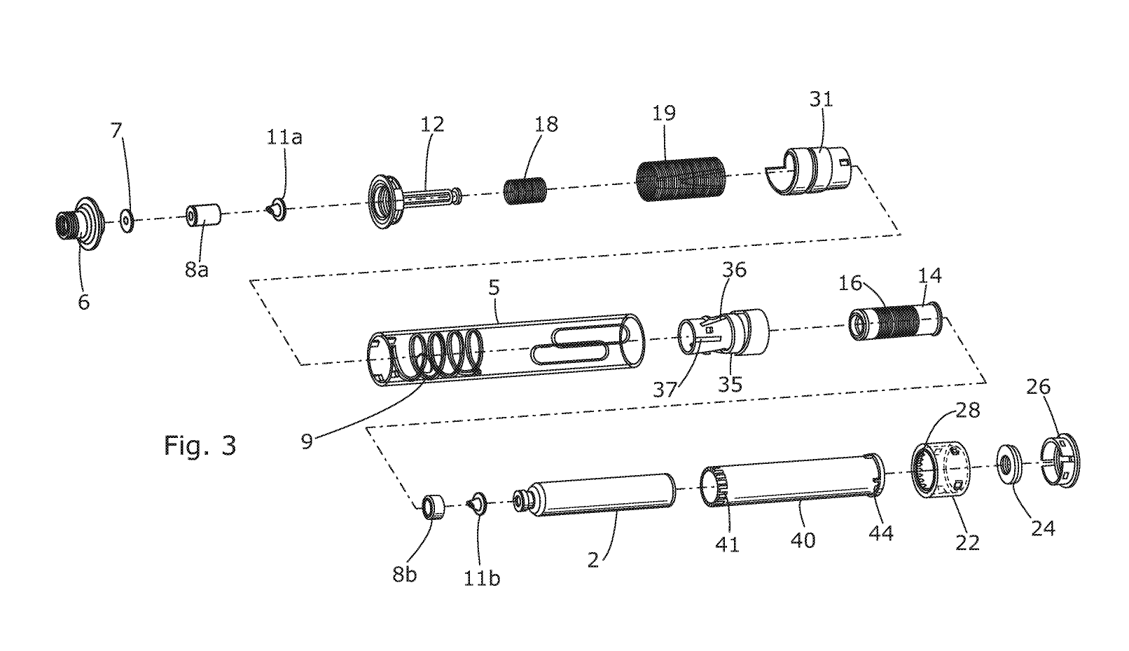

Referring initially to FIGS. 1 to 3, injection device 1 according to an embodiment of the invention comprises a pen-type injector which is intended to allow a user to select a dose magnitude, offer up the injection device to the skin, manually insert a needle to the required depth and then trigger the device to cause the selected dose to be introduced into the injection site through the needle.

The injecting device 1 comprises a main housing 5 within which is retained a cartridge 2 of medicament (although it will be understood that in some arrangements, for example a disposable injection device, the cartridge 2 could be integrally formed within the device). The housing also contains a delivery mechanism 10 and a dose-setting arrangement 30. The forward end of the injection device is provided with a needle receiving portion 6 onto which a single-use needle tip (typically a double-ended needle tip which would pierce the septum 7) can be screwed prior to use. The rearward end of the injection device 1 is provided with a button assembly 20 which, as will be described in further detail below, may be rotated to allow a dose to be selected and urged forwardly to deliver a dose of medicament from the cartridge or syringe 2.

The delivery mechanism 10 comprises a piston 12 which is angularly and axially fixed within the housing 5 and which is provided with a conduit for the delivery of medicament running through its centre. Rearwardly of the piston 12, a chamber 14 is provided which extends from a forward end which surrounds and sealingly engages with the rearward end of the piston 12 to a rearward end which is provided with a seat 17 for receiving the forward end of the cartridge 2. A pair of valves 11 a and 11 b are provided at the forward and rearward end of the conduit formed by the piston 12 and chamber 14 and are each retained by an annular retaining member 8a and 8b. Each valve 11a and 11b is a non-return valve which only allows medicament to flow through the injection device in the forward direction. As the chamber 14 is axially moveable within the housing 5 (but is provided with a latch arrangement to engage the shaft of the piston 12 and prevent the piston from disengaging from the chamber 14) the total volume of the conduit formed in the delivery mechanism 10 may be varied. As a result of the positioning and direction of the non-return valves 11 a and 11 b, it will be appreciated that forward movement of the chamber 14 relative to the piston 12 (i.e. reducing the volume of the conduit) will result in fluid being expelled from the delivery mechanism 10 via valve 11 a and the needle receiving portion 6. Conversely, rearward movement of the chamber 14 relative to the piston 12 (increasing the volume of the conduit) will produce a negative pressure within the conduit and act to draw medicament from the syringe 2 and will draw the bung 3 of the cartridge forward. A return spring 18 may be provided to bias the piston 12 and chamber 14 towards the expanded (primed) position.

A drive shaft 40 extends between the actuation button 20 and the housing 5 and (as will be described below) engages the intermediate collar 35 of the dose selection arrangement 30. The drive shaft 40 surrounds and encloses the cartridge 2. The drive shaft may 40 may be removably mounted to the dose selection arrangement 30 (for example by a snap or push fit) such that the cartridge 2 may be accessed and replaced. Alternatively, the cartridge 2 may be permanently attached within the device (for example in a disposable device).

The dose-setting arrangement 30 comprises a threaded annular member 31 and an intermediate collar 35. The threaded member 31 is positioned within the housing 5 and generally surrounds the delivery mechanism 10. The threaded member 31 engages a thread 9 which is associated with (and typically moulded into the internal surface of) the housing 5. The intermediate collar 35 is located within the thread member 31 and externally to the chamber 14 of the delivery mechanism 10. It will be appreciated that as a result of the threaded engagement between the housing 5 and the threaded member 31 relative axial movement between the threaded member 31 and the housing 5 will result in angular rotation of the threaded member 31 (and vice versa). The forward end of the threaded member 31 is provided with a stop 32 which limits the forward movement of the threaded member relative to the housing 5 (which may also be provided with a corresponding stop). The stop may be provided by a blind end 9a to the thread 9 of the housing 5. A helper spring 18 may be provided to assist the movement of the threaded member 31 through the housing 5 (although alternatively the helix angle of the threaded engagement may be adjusted to decrease the resistance to movement).

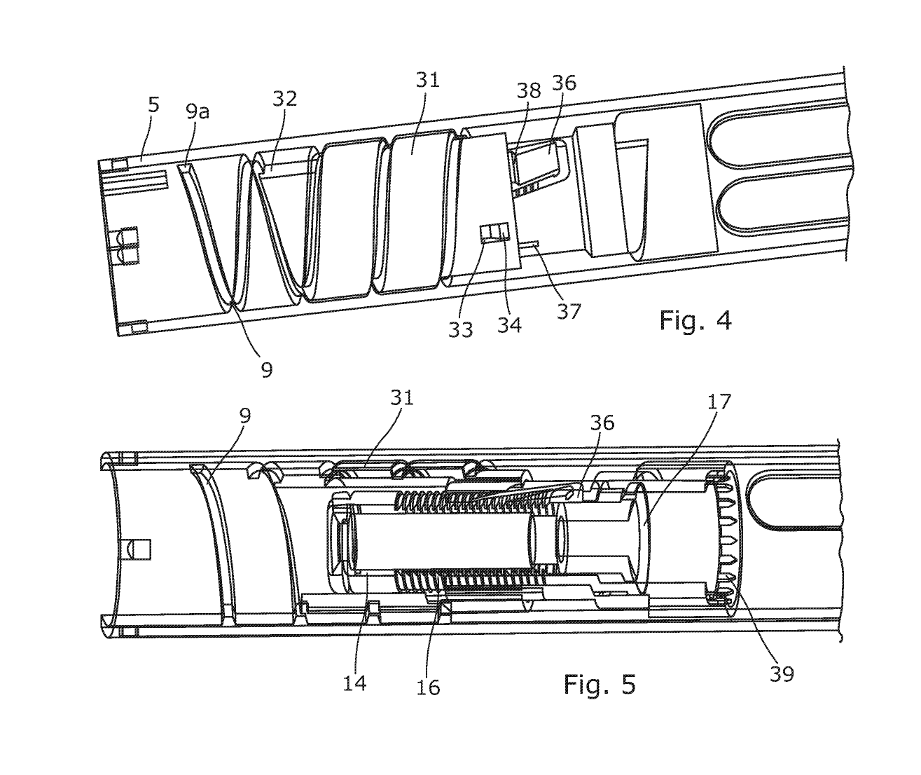

As shown in further detail in FIGS. 4 and 5, the intermediate member 35 is angularly fixed relative to the threaded member 31 by virtue of a pair of diametrically opposed radially outwardly extending splines 37 which engage a corresponding keyway within the inner surface of the threaded member 31. Additionally, relative axial movement between the intermediate collar 25 and threaded member 31 is limited by a radially extending protrusion 34 on the intermediate collar 35 which is received in a window 33 formed in the threaded member 31. However, it will be noted that the radial protrusion 34 is provided with a ramped forward surface such that under forward directed load the protrusion 34 may disengage the window 33 and allow a limited degree of forward axially movement of the intermediate member 35 relative to the threaded member 31. In contrast, the rearward face of the radial protrusion 34 is provided with a stepped profile such that upon (return) rearward axial movement the intermediate collar 35 will be axially restrained relative to the threaded member 31 by the radial protrusion 34 returning into engagement with the window 33.

Mutual engagement features are arranged between the chamber 14 of the dose delivery mechanism 10 and the intermediate collar 35 of the dose setting arrangement 30. On the chamber 14 the engagement feature simply comprises a series of circumferential ribs 16 provided along the axial length of the external surface of the chamber 14. On the intermediate collar 35 the engagement feature comprises a pair (although any number may be provided) of diametrically opposed resilient fingers 36. The resilient fingers 36 extend axially rearwardly from the body of the intermediate collar 35 and are cantilevered at their forward end. The resilient fingers 36 are provided with radially inwardly directed tabs at their free end which are dimensioned to engage the ribs 16 of the chamber 14. The resilient fingers 36 are biased to a radially outward position in which they do not engage the ribs 16 and may be resiliently deformed inwardly into an engaged position. The exterior of the resilient fingers 36 are provided with an radially outwardly extending protrusion 38 which acts as a cam against the inner surface of the threaded member 35 (which may be provided with a suitably ramped cam surface) to urge the resilient fingers inwardly when the intermediate collar slides axially forward and into the threaded member 35.

In use, the user places the injection device 1 into a dose setting arrangement by drawing the button assembly 20 rearwardly such that rearwardly facing splines 28 positive mesh with the forward facing splines 44 at the rearward end of the drive shaft 40. In this position the button assembly 20 is positively angularly coupled to the drive shaft 40. The user rotates the button assembly 20 which transmits the angular rotation via inter-engaging splines 42 (at the forward end of the drive shaft 40) and 39 (at the rear end of the intermediate collar 35) to the intermediate member 35. As the intermediate member 35 is angularly fixed relative to the threaded member 31 the rotation is also transmitted to the threaded member 31 causing it to axially cam along the thread 9 of the housing 5. During this operation the resilient fingers 36 of the intermediate member 35 are un-deflected and do not engage the ribs 16 of the chamber 14. Thus, the axial starting position of the dose setting arrangement 30 may be set without adjusting the position of the dose delivery mechanism 10. In order to enable the user to quantify the dose being selected a window (not shown) may be provided in the housing 5 and incremental dose indications may be provided on the threaded member 31 such that the dose may be aligned with the window.

Once the selected dose has been “dialled in” the user inserts an attached needle into an injection site and urges the button assembly 20 forwardly. The forward force on the button assembly 20 causes the clutch arrangement of the button to disengage. This frees drive shaft 40 to rotate angularly relative to the button 20 which would be engaged by the user's finger (and a bearing 24 is provided within the button assembly to assist this free rotation). The forward force on the drive shaft 40 axially moves the the drive shaft 40 into the housing 5. The forward end of the drive shaft 40 abuts the rearward end of the intermediate collar 35 such that both are urged forwards. Due to the ramped forward profile of the projections 34 the axial coupling between the intermediate collar 35 and threaded member 31 is disengaged allowing the intermediate collar 35 to slide forwardly within the threaded member 31 (until the rearward end of the threaded member 31 engages a shoulder on the exterior of the collar 35). The relative sliding action moves the resilient fingers 38 under the threaded member 31 with the projection 38 and ramped surface camming the fingers radially 38 inwardly. Thus, the fingers engage the ribs 16 of the chamber 14 so as to axial couple the dose setting arrangement 30 and dose delivery mechanism 10. As the drive shaft 40 and dose setting arrangement 30 continue to be urged forwards, the chamber 16 is also carried forward such that the piston 12 telescopes into the chamber 16 and reduces the volume of the conduit to expel a dose of medicament. As the ribs 16 are circumferential the dosing arrangement 30 (along with the drive shaft 40) are free to rotate relative to the chamber 14 and will do so as a result of the engagement between the threaded member 31 and thread 9 on the housing 5. Once the threaded member 31 reaches the end of the thread 9a the dose setting arrangement is no longer able to rotate and thus limits the forward movement of the chamber 16.

It will, thus, be appreciated that the initial axial position selected during the dose setting action will determine the axial position at which the dose setting arrangement 10 will engage the ribs 16 of the chamber 14 and will thereby set the axial movement of the chamber 14 which is possible before the threaded member 31 reaches the forward stop.

Upon release of the pressure from the button 20 the return spring 18 will urge the chamber 14 rearwardly towards its initial (primed) position. The intermediate collar 35 is able to move rearwardly relative to the threaded collar 31 until the radial projections 34 snap back into the windows 33 and prevent further rearward relative movement (due to the step rearward profile of the projections 34). The arrangement of the dose delivery mechanism 10 and dosing selection arrangement, advantageously, allows the delivery mechanism to return to its rearmost position after each use unless there is insufficient medicament remaining in the cartridge 2, when the delivery mechanism will travel back until the delivery chamber is filled.

As the chamber 14 will not return to its fully rearward position when there is insufficient medicament remaining in the cartridge 2 (since it is unable to draw any further medicament from the cartridge) it will stop in a partially rearward position. An engagement feature, for example a helical tooth, may be provided between the chamber 14 and the dosing mechanism (in particular the intermediate collar 35). The engagement feature is arranged to ensure that the range of movement of the intermediate collar 35 is limited relative to the rearward end of the chamber 14. Since the dose is set when the device is primed and the chamber is rearward this means that the movement of the collar 35 will only be limited when the chamber 14 is unable to return fully rearwardly (i.e. when there is insufficient medicament remaining). Therefore, the maximum dose that the user is able to set may be automatically varied and the user may be prevented from setting a dose which exceeds that available in the injector.

Although the invention has been described above with reference to a preferred embodiment, it will be appreciated that various changes or modifications may be made without departing from the scope of the invention as defined in the appended claims.

1. An injection device apparatus for use with a cartridge or syringe to deliver a plurality of doses of medicament therefrom, the injection device comprising:
a housing;
an axially moveable dose delivery mechanism arranged to deliver a dose from said cartridge or syringe;
an actuation button for imparting movement to said delivery mechanism; and
a dose setting arrangement to select a dose volume, wherein the dose setting arrangement comprises:
a threaded member arranged to engage a formation on the housing such that axial movement relative to the housing results in relative angular movement of the threaded member;
a stop, fixed relative to the housing, and arranged to cooperate with the threaded member limiting axial movement thereof; and
complementary engagement features moveable between an engaged position in which relative axial movement of the delivery mechanism and threaded member is prevented, and a disengaged position in which the threaded member may be moved relative to the delivery mechanism to adjust the dose, and wherein forward movement of the actuation button moves the engagement features into the engaged position.

2. The injection device as claimed in claim 1, wherein the complementary engagement features are biased towards the disengaged position.

3. The injection device as claimed in claim 1, wherein a first of the complementary engagement features comprise radially moveable finger associated with at least one of the delivery mechanism or the dose setting arrangement.

4. The injection device as claimed in claim 3, wherein a second of the complementary engagement features comprise a plurality of axially spaced projections associated with the other of the delivery mechanism or the dose setting arrangement.

5. The injection device as claimed in claim 1, wherein the complementary engagement features comprise a plurality of axially spaced ribs on a shaft of the delivery mechanism and a moveable finger attached to the dose setting arrangement and arranged to selectively engage one of the ribs.

6. The injection device as claimed in claim 1, wherein the dose setting arrangement further comprises an intermediate member in communication with the threaded member and the delivery mechanism.

7. The injection device as claimed in claim 6, wherein the intermediate member comprises the complementary engagement features for engaging the delivery mechanism.

8. The injection device as claimed in claim 7, wherein the intermediate member is angularly fixed relative to the threaded member.

9. The injection device as claimed in claim 7, wherein the intermediate member is axially moveable relative to the threaded member.

10. The injection device as claimed in claim 9, wherein axial movement of the intermediate member relative to the threaded member moves the complementary engagement features to the engaged position.

11. The injection device as claimed in claim 10, wherein one of the complementary engagement features or threaded member is provided with a cam surface.

12. The injection device as claimed in claim 1, wherein the delivery mechanism comprises a plunger, the plunger being provided with complementary engagement features.

13. The injection device as claimed in claim 1, wherein the delivery mechanism comprises a displaceable chamber or piston.

14. The injection device as claimed in claim 1, wherein the delivery mechanism is arranged to draw medicament from the cartridge or syringe by negative pressure.

15. The injection device as claimed in claim 1, further comprising a drive member which extends axially between the actuation button at a rearward end and the delivery mechanism at a forward end.

16. The injection device as claimed in claim 15, wherein the drive member is axially and angularly coupled to the dose setting arrangement.

17. The injection device as claimed in claim 16, wherein the actuation button is connected to the drive member via a clutch such that the actuation button may be selectively angularly coupled to the drive member.

18. The injection device as claimed in claim 17, wherein forward movement of the actuation button disengages the clutch.

19. An injection device apparatus for use with a cartridge or syringe to deliver a plurality of doses therefrom, the injection device comprising:
a housing;
a dose delivery mechanism arranged to deliver a dose from said cartridge or syringe;
an actuation button for imparting actuation movement to said delivery mechanism; and
a dose setting arrangement to select a dose volume, wherein the actuation movement is transferred from the actuation button to the dose setting arrangement and the resulting axial movement of the dose setting arrangement causes inter-engagement with the dose delivery mechanism to cause delivery movement thereof.
